(19) United States
(12) Patent Application Publication
(10) Pub. No.: US 2018/0369609 A1
(43) Pub. Date: Dec. 27, 2018

(54) Medical Devices with Laser Therapy Capability

(71) Applicants: The Regents of the University of California, Oakland, CA (US); Arizona Board of Regents on Behalf of the University of Arizona, Tucson, AZ (US)

(72) Inventors: Petra Beate Wilder Smith, San Juan Capistrano, CA (US); Rongguang Liang, Tucson, AZ (US); Cherie Yvette Wink, Yorba Linda, CA (US)

(73) Assignees: The Regents of the University of California, Oakland, CA (US); Arizona Board of Regents on Behalf of the University of Arizona, Tucson, AZ (US)

(21) Appl. No.: 16/116,377

(22) Filed: Aug. 29, 2018

Related U.S. Application Data
(63) Continuation-in-part of application No. 15/802,915, filed on Nov. 3, 2017.
Application No. 15/802,915 is a continuation of application No. PCT/US2016/031071, filed on May 5, 2016.
(60) Provisional application No. 62/551,579, filed on Aug. 29, 2017.
(60) Provisional application No. 62/157,452, filed on May 5, 2015.

(51) Int. Cl. A61N 5/06; A61C 1/00; A61C 1/07; A61C 3/03; A61C 17/20; A61B 17/14; A61B 17/16; A61B 17/32; A61C 3/06

(52) U.S. Cl. CPC A61N 5/0624; A61C 1/0046; A61C 1/07; A61C 1/0023; A61C 1/0061; A61C 3/03; A61N 2005/067; A61B 17/14; A61B 17/1671; A61B 17/320068; A61C 3/06; A61B 2017/320024; A61C 17/20

(57) ABSTRACT

Various types of medical devices incorporating a light therapy system are disclosed. In at least one embodiment, the medical device provides an ultrasonic driver and a light therapy driver. The ultrasonic driver provides an ultrasonic vibration signal output port and is configured to discharge an ultrasonic frequency vibration signal therefrom. The light therapy driver provides a laser light output port and is configured to discharge laser light therefrom. A handpiece housing of the medical device provides an input assembly connected to each of the ultrasonic vibration signal output port and laser light output port. The input assembly provides an ultrasonic tip assembly configured to output each of the vibration signal and laser light at an operational end of the tip assembly. Thus, the vibration signal and laser light are capable of being simultaneously or alternatingly delivered via the tip assembly.

### RELATED APPLICATIONS

This application claims priority and is entitled to the earliest effective filing dates of U.S. provisional application Ser. No. 62/551,579, filed on Aug. 29, 2017, and U.S. non-provisional application Ser. No. 15/802,915, filed on Nov. 3, 2017.

This is a continuation-in-part application and so claims the benefit pursuant to 35 U.S.C. § 120 of a prior filed and co-pending U.S. non-provisional patent application Ser. No. 15/802,915, filed on Nov. 3, 2017, which is a continuation of international application number PCT/US2016/031071, filed on May 5, 2016, which claims priority to U.S. provisional application Ser. No. 62/157,452, filed on May 5, 2015. This application also claims priority pursuant to 35 U.S.C. § 119(e) and is entitled to the filing date of U.S. provisional patent application Ser. No. 62/551,579, filed on Aug. 29, 2017. The contents of the aforementioned applications are incorporated herein by reference.

### BACKGROUND

The subject of this patent application relates generally to dental scalers, air polishing units, dental handpieces (e.g., for scaling, cutting, grinding, or polishing), air abrasion instruments, piezoelectric endodontic instruments, bone saws (e.g., piezoelectric and reciprocating), bone drilling instruments, general surgery handpieces, high-torque surgical power tools, morcellator systems (e.g., laparoscopic surgical instruments), craniotome drills, medical shaver (e.g., angled and straight shavers), and spinal surgery handpieces (e.g., angled handpiece) with light therapy functionality such as laser bacterial load reduction capability.

Applicant(s) hereby incorporate herein by reference any and all patents and published patent applications cited or referred to in this application.

By way of background, Currently, medical and dental procedures include cutting, grinding, scaling, polishing and other similar procedures that involve the use of reciprocating, rotating, vibrating or otherwise moving working instruments that come in contact with patients' anatomy and biological tissues and other matter. These instruments can dislodge and spread small particles of matter which can linger in the surrounding surgical field and can be come suspended in the air surrounding the surgical field. For example, when cutting through a bone using a periodontal hypersonic or reciprocating bone saw of a type commonly used in dental and other forms of surgery, microscopic particles of bone and blood can be dislodged from the patient and broadcast into the air. Bacteria and other contaminants that reside on the tissue can spread through other portions of the patient's body and the surgical environment. These contaminants are a potential source of infection to patients as well as health care providers.

Currently, nonsurgical periodontal therapy, including scaling and root planning, as well as periodontal scaling with root debridement involves a series of instrumentation procedures. Aerosol produced during the use of power scalers has droplet nuclei particles which linger in the environment for extended periods of time, and is a potential source of infection to patients as well as oral health care providers. The release of an increased bacterial load into the oral cavity may result in the spread of periodontal and oral pathogens.

Laser Bacterial Reduction (LBR) and other disinfecting treatments of a patient tissue site can be performed prior to any dental or medical procedures that dislodge small particles of patient tissue to prevent the spread of pathogenic bacteria from a diseased patient site, such as an oral cavity or other patient tissue site. Clinicians can then proceed to use working instruments, such as an Ultrasonic or Piezoelectric scaler, bone saw or other working instrument, to perform medical or dental procedures such as, removing calcified deposits while simultaneously disrupting the plaque biofilm and cutting through patient bone. In the case or dental scalers, the more intricate work of scaling and root planning, utilizing a series of manual curettes, then follows. Once the dental or medical procedure has been performed, the clinician may choose to utilize the laser again to perform soft tissue curettage or other disinfecting treatments of the tissues.

Scaling and root planning, also known as conventional periodontal therapy, non-surgical periodontal therapy, or deep cleaning, is the process of removing or eliminating the etiologic agents, dental plaque, its products, and calculus. Periodontal scalers and periodontal curettes are used for such procedures.

An ultrasonic scaler is a tool which utilizes various tips for supplying high-frequency vibrations to the tooth surfaces for the purpose of removal of adherent deposits and bits of inflamed tissue from the inner walls of the gingival sulcus or periodontal pocket. Mechanical root debridement results in a smear layer containing bacteria, bacterial endotoxins, and contaminated root cementum and usually does not remove plaque and calculus completely from interradicular septa and root concavities. Thus, a significant disadvantage of ultrasonic scalers, for the patient and the clinician, is the formation of a contaminated aerosol. Similarly, other working instruments for dental and medical procedures also have the significant disadvantage of dislodging small particles of patient tissue, including in aerosol form. Moreover, the working instruments can expose and/or dislodge tissue during the procedure that was not previously accessible to disinfecting treatments.

In recent years, the use of lasers in dentistry and medical procedures has continued to expand. Laser systems are cleared for marketing in the United States via the Food and Drug Administration (FDA) Premarket Notification (510(k)) process. The applications of lasers in dentistry and medicine include sulcular debridement, laser curettage, laser-assisted new attachment procedure (LANAP), reduction of bacteria levels in periodontal pockets (or pocket sterilization) referred to as laser bacterial reduction (LBR), laser-facilitated wound healing, and laser root planning. For example, erbium-doped: yttrium, aluminum, and garnet (Er:YAG) laser radiation has been suggested as an alternative instrumentation modality for the treatment of chronic periodontitis. Dental hygienists use lasers for laser bacterial reduction, laser curettage, intrasulcular debridement in scaling and root planning procedures, aphthous ulcer removal, and pit and fissure sealants. Periodontists use lasers for osseous surgery and to correct osseous defects, gingivectomies, frenectomies, gingival curettage, implant placement, and soft tissue crown lengthening. Surgeons use lasers to cut, ablate and cauterize tissues, diagnose disease, treat cancer, repair patient tissue, and perform surgeries in both invasive and noninvasive applications.

Currently, periodontal probes, ultrasonic scalers, curettes, and dental lasers, each have their own application and working tip. Dental professionals measure the sulcus or periodontal pocket prior to instrumentation utilizing a periodontal probe to assess the geography. Prior to any instrumentation, the clinician may perform laser bacterial reduction. Next, an ultrasonic scaler can be used, followed by the use of curettes, to remove deposits from the tooth surfaces. Dental lasers can then be used again to remove the remaining soft tissue tags, continue reduction of bacterial levels, and possibly promote wound healing.

Time is taken away from patient care each time the clinician has to change instruments and switch back and forth between the periodontal probe, ultrasonic scaler, curette, and laser. Thus there remains a need in the art for a new device that combines these individual steps while promoting a potential healthier environment, and reducing the risk of disease transfer, both inside and outside of the oral cavity.

Furthermore, other dental and medical procedures using working instruments require the clinician to pause the procedures at various times to perform cleaning and disinfecting activities at the surgical site. This lengthens the time for the completion of dental and medical procedures. Moreover, the exposure of tissue that was previously accessible to disinfecting treatments raises the risk of spreading contaminated tissue during intervals between disinfecting. Thus, there remains a need in the art for new devices that combine the steps of disinfecting and the use of working instruments while promoting a potentially healthier environment, and reducing the risk of disease transfer, both inside and outside of the patient.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUM MARY

Aspects of the present invention teach certain benefits in construction and use which give rise to the exemplary advantages described below.

Infection control is a constant and critical part of all dental and medical procedures. Because dental and medical working instruments can generate a significant amount of tissue dislodgement (e.g., aerosol and/or splatter due to vibration scaling, blowing, cutting, grinding, drilling, polishing and/or shaving) use of various bacterial and other contaminant reducing (disinfecting) procedures is recommended. Ultimately, dental and medical professionals are exposing themselves and their patients to potentially pathogenic tissue. Thus a device that combines the functionality of various dental and medical working instruments and laser bacteria load reduction can reduce the number of pathogenic microbes from becoming airborne and potential reduce the amount of cross-contamination within the body as the instruments are taken from site to site.

An aspect of at least one of the inventions disclosed herein, includes the realization that a dental or medical working instrument can be modified to include a passage allowing light to travel through the working instrument assembly through a distal end of the working instrument assembly in the vicinity of operational end of the instrument assembly which can be contacted with a patient's anatomy (e.g., a patient's teeth, gums, tissue or bone). For example, a proximal end of a vibrating, liquid or gas blowing, reciprocating, and/or rotating portion of the instrument assembly can include an input opening configured to receive light from a light source, such as a laser light source, and an output opening on a distal portion of the vibrating, liquid or gas blowing, reciprocating, and/or rotating portion. The output opening can be disposed in the vicinity or at the distal-most portion of the instrument assembly. Thus, during use, light having an optical strength sufficient for bacteria load reduction can be directed towards patient tissue and any present contaminants and thus treated with the bacteria load reducing light during dental or medical procedures (e.g., scaling, blowing, cutting, grinding, drilling, polishing and/or shaving). Thus, the bacteria load can be reduced at the point of and simultaneous with the use of the working instrument assembly.

An aspect of at least one of the inventions disclosed herein, includes the realization that a dental scaler tip assembly can be modified to include a passage allowing light to travel through the scaler tip assembly through the distal end of the tip assembly in the vicinity of operational end of the scaler tip assembly which can be pressed against deposits along patient's anatomy, such as on a patient's teeth and/or gums. For example, a dental scaler tip assembly can include a channel with an input opening configured to receive light from a light source, such as a laser light source, and an output opening on a distal portion of the tip assembly. The output opening can be disposed in the vicinity or at the distal-most portion of the scaler tip assembly. Thus, during use, light having an optical strength sufficient for bacteria load reduction can be directed towards deposits to be removed with the scaler during a procedure and thus treated with the bacteria load reducing light during scaling, or other procedures. Thus, the bacteria load can be reduced at the point of and simultaneous with the use of the scaler tip assembly.

Another aspect of at least one of the inventions disclosed herein includes the realization that using prior art techniques, such as the use of a separate laser bacteria load reducing technique prior to scaling is that such laser based bacteria load reducing techniques are limited in the depth to which the bacteria load is reduced at the deposit or anatomy. Thus, if a first bacteria load reducing technique is applied to a patient, then a scaling operation is performed, additional untreated bacteria can be uncovered during the course of the scaling procedure, thereby increasing the risk of aerosolizing untreated bacteria after having been uncovered during a scaling procedure.

Thus, an aspect of at least one of the inventions disclosed herein includes the realization that including a light discharge functionality with a scaler tip assembly provides the additional benefit of the ability to reduce the bacterial load of untreated bacteria contemporaneously uncovered during a scaling procedure.

In some embodiments, an ultrasonic scaler guides laser light to the tip of the scaler. As noted above, in some known prior art ultrasonic scalers, the traditional ultrasonic insert has only one function, which is to remove hard and soft deposits along with extrinsic stain, it does not contain a laser light.

Thus, in some embodiments, a scaler device can include an insert or attachment configured to guide laser light from a handheld portion to the tip of the scaler through a hollow canal. Thus, only one device is needed for the utilization of the ultrasonic scaler and dental laser. Such a device can reduce a procedure time significantly and also reduce the cost.

In some embodiments, ultrasonic insert can also guide the water to the tip of the scaler through a hollow canal.

In some embodiments, a tip of an ultrasonic scaler can be color coded, similar to that of a periodontal probe, to allow for measurement and reference as it is used. Additionally, a plurality of ultrasonic scaler tips having different sizes and color coded according to their different sizes can be packaged together in a kit.

Infection control is a constant and critical part of all dental hygiene procedures. Because ultrasonic instruments can generate a significant amount of aerosol and splatter due the rapid vibration and water spray, use of the high speed evacuation is recommended by the Occupational Health and Safety Administration (OSHA). Without an assistant, many clinicians find themselves unable to adapt the high speed evacuation to where they are working with one hand, so frequently, they will settle for use of the slow speed suction because of its ease of use, despite the current recommendations. Ultimately, dental clinicians are exposing themselves and their patients to potentially pathogenic aerosol. Thus a device that combines ultrasonic scaling functionality and laser bacteria load reduction can reduce the number of pathogenic microbes from becoming airborne and potential reduce the amount of cross-contamination within the mouth as the instrument is taken from site to site.

In some embodiments, an ultrasonic scaler can be configured to work with different laser sources. In such configurations, additional components can be unnecessary to accommodate different laser sources.

Another aspect of at least one of the inventions disclosed herein includes the realization that dental procedures, such as scaling, can be combined with measurement. For example, procedures such as scaling involve a practitioner moving a scaler device carefully over the surface of patient anatomy, and optionally using magnification to assist in visualizing the procedural field. The procedure such as scaling is also procedurally similar to probing, for example, inspecting patient's anatomy for defects and the measurements of the size of such defects. During the movements commonly used in scaling procedures, a practitioner can move the tip of a scaler assembly into close proximity and/or contact with a defect.

An aspect of at least one of the inventions disclosed herein includes the realization that dental scaler tip assemblies can be modified to simplify a process for measurement and/or estimation of measurements of dental defects, for example, with reference indicia. For example, a dental scaler tip assembly with reference indicia (such as color coding) can provide a reminder to a practitioner as to a dimension of a portion of the dental scaler tip assembly. For example, in some embodiments, a referenced dimension would be a maximum width of a distal tip of a dental scaler tip assembly.

Thus, during a procedure such as a scaling procedure, as a practitioner moves the dental scaler tip assembly around the patient's anatomy, the practitioner can estimate the size of anatomical features and/or defects with visual reference to the referenced dimension of the dental tip assembly.

Another aspect of at least one of the inventions disclosed herein includes the realization that if a plurality of dental scaler tip assemblies are packaged together in a kit having a predetermined distribution of sizes of a referenced dimension, such as the largest width of a distal tip of such scaler tip assemblies, a practitioner can more easily measure or estimate a size of anatomical features and/or defects of patient anatomy during use. For example, a dental scaler tip kit can include a plurality of differently sized dental scaler tip assemblies, which can be color coded, so that a practitioner can readily identify a size of the referenced dimension of the scaler tip assembly to further simplify a process for measuring or estimating a dimension of an anatomical structure or defect.

In some embodiments, a dental scaler system can comprise an ultrasonic driver having an ultrasonic vibration signal output port, the ultrasonic driver configured to discharge an ultrasonic frequency vibration signal from the ultrasonic vibration signal output port. A laser light source can have a laser light output port; the laser light source configured to discharge laser light from the laser light output port. A hand-piece housing can have an outer surface configured to be graspable and manipulable with a user's hand, the hand-piece housing having an input assembly connected to the ultrasonic vibration signal output port so as to receive an ultrasonic vibration signal from the ultrasonic driver, the input assembly also connected to the laser light output port so as to receive laser light from the laser light source, the hand-piece also comprising a output assembly configured to output an ultrasonic vibration and laser light. An ultrasonic scaler member can have a proximal end and a distal end, the proximal end of the ultrasonic scaler member can be connected to the output assembly of the handpiece housing, the ultrasonic scaler member comprising a light guide extending from a light guide input at the proximal end of the ultrasonic scaler member to a light guide output at the distal end of the ultrasonic scaler member, the light guide output configured to discharge laser light from the distal end of the ultrasonic scaler member.

In some embodiments, the light guide is configured to receive laser light having a wavelength in the range of 0.4 μm to 3.0 μm.

In some embodiments, the light guide comprises a hollow passage extending from the proximal end of the ultrasonic scaler member to the distal end of the ultrasonic scaler member, the light guide comprising an inner surface with high reflectivity.

In some embodiments, the handpiece housing comprises a light coupling including a reflector, connecting the input assembly with the output assembly.

In some embodiments, the light coupling comprises a fiber coupler.

In some embodiments, the ultrasonic scaler member includes a concave portion, and water outlet port being disposed in the concave portion.

In some embodiments, the ultrasonic scaler member comprises a canal extending from the proximal end to the distal end of the ultrasonic scaler member, the canal configured to guide water from the proximal end to the distal end.

In some embodiments, a dental scaler can comprise a hand-piece housing having an outer surface configured to be graspable and manipulable with a user's hand. An ultrasonic scaler member can have a proximal end and a distal end, the proximal end of the ultrasonic scaler member being connected to the hand-piece housing, the proximal end of the ultrasonic scaler member including a light input portion and a light guide extending from the light input portion to a light output portion at a distal end of the ultrasonic scaler member, the light output portion being configured to discharge laser light from the distal end of the ultrasonic scaler member.

In some embodiments, an ultrasonic transducer can be disposed in the hand-piece and in vibrational communication with the ultrasonic scaler member, the ultrasonic transducer configured to vibrate the ultrasonic scaler member at an ultrasonic frequency.

In some embodiments, an ultrasonic driver can be operationally connected to the ultrasonic scaler member and configured to transfer an ultrasonic frequency vibration signal to the ultrasonic scaler member.

In some embodiments, a laser light source can be operationally connected to the ultrasonic scaler member and configured to provide laser light to the ultrasonic scaler member.

In some embodiments, an input device can be disposed on ab outer surface of the hand-piece housing configured to control discharge of light through the ultrasonic scaler member.

In some embodiments, the light guide is configured to receive laser light having a wavelength in the range of 0.4 μm to 3.0 μm.

In some embodiments, wherein the light guide has an upstream end and a downstream end, the upstream end being larger than the downstream end.

In some embodiments, the light guide has an inner diameter that gradually changes from a larger diameter at the upstream end to a smaller diameter at the downstream end.

In some embodiments, the handpiece housing comprises a light coupling including a reflector, connecting the input assembly with the output assembly.

In some embodiments, wherein the light coupling comprises a fiber coupler.

In some embodiments, wherein the ultrasonic scaler member includes a concave portion, and water outlet port being disposed in the concave portion.

In some embodiments, wherein the ultrasonic scaler member comprises a canal extending from the proximal end to the distal end of the ultrasonic scaler member, the canal configured to guide water from the proximal end to the distal end.

In some embodiments, a dental scaler tip member can comprise a proximal end and a distal end, the proximal end of the dental scaler member being configured to be connectable to an ultrasonic scaler hand-piece housing, the proximal end of the ultrasonic scaler member including a light input portion and a light guide extending from the light input portion to a light output portion at a distal end of the ultrasonic scaler member, the light output portion being configured to discharge laser light from the distal end of the ultrasonic scaler member.

In some embodiments, the light guide has an inner surface with a reflectivity of at least 50%.

In some embodiments, the light guide is configured to guide laser light having a wavelength in the range of 0.4 μm to 3.0 μm from the proximal end to the distal end of the dental scaler tip member.

In some embodiments, the dental scaler tip member is configured to be vibrated at an ultrasonic frequency during a dental scaling procedure.

In some embodiments, a dental scaler tip kip can comprise at least first and second dental scaler tip members, each of the plurality of dental scaler tip members comprising a proximal end and a distal end, the proximal end of each dental scaler member being configured to be connectable to an ultrasonic scaler hand-piece housing, the distal end of each of the dental scaler tip members having a different dimension, each of the dental scaler tip members having a different color, and all of the plurality of dental scaler tip members being contained in a single container.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a schematic diagram of prior art ultrasonic dental scaler, in accordance with at least one embodiment;

FIG. 2 is a schematic diagram of a prior art laser curettage device, in accordance with at least one embodiment;

FIG. 3A is a schematic diagram of an embodiment of a scaler system with light therapy functionality including a sonic driver unit and a light driver unit, both of which are connected to an embodiment of a scaler handheld piece and an embodiment of a dental scaling tip assembly, in accordance with at least one embodiment;

FIG. 3B is a schematic illustration of a further embodiment of the dental scaler system with light therapy functionality, including an integrated sonic and light driver and an integrated connector between the driver and handheld piece, in accordance with at least one embodiment;

FIG. 3C is a schematic diagram of the connections between the integrated driver, handpiece, and tip assembly of the embodiment of FIG. 3B, in accordance with at least one embodiment;

FIG. 4 is a schematic diagram of a dental scaler assembly including a handheld piece, a driver connector, and a scaler tip assembly in which light is transmitted to the handheld piece, and into the scaler tip assembly, and discharged through a distal end of the scaler tip assembly, in accordance with at least one embodiment;

FIG. 5 is a modification of the embodiment of FIG. 4, including a water discharge opening in a concave area of the dental tip assembly, in accordance with at least one embodiment;

FIG. 6 is a schematic illustration of a further modification of the embodiments of FIGS. 4 and 5 in which an optical fiber extends through the handheld piece and the dental scaler tip assembly, in accordance with at least one embodiment;

FIG. 7 is a schematic diagram of a further modification of the embodiments of FIGS. 4-6 in which a fiber is used for directing light to the handheld piece, the handheld piece and a portion of the dental scaler tip assembly includes an optical passage and a distal portion of the scaler tip assembly includes an additional fiber for directing light to a distal end of the tip assembly, in accordance with at least one embodiment;

FIG. 8 is a schematic illustration of yet another modification of the embodiments of FIGS. 4-7, including a light input port on the handheld piece, separate from the connector to the driver to the sonic driver unit, and including a reflector for directing the light parallel to the water channel, in accordance with at least one embodiment;

FIG. 9 is a schematic diagram of a kit including plurality of color-coded dental scaler tip members with different dimensions, in accordance with at least one embodiment;

FIG. 10 is a schematic diagram of an air polishing unit, in accordance with at least one embodiment;

FIG. 11 is a schematic diagram of a dental handpiece, in accordance with at least one embodiment;

FIG. 12 is a schematic diagram of a dental handpiece, in accordance with at least one embodiment;

FIG. 13 is a schematic diagram of a dental abrasive tool, in accordance with at least one embodiment;

FIG. 14 is a schematic diagram of an endodontic instrument, in accordance with at least one embodiment;

FIG. 15 is a schematic diagram of a dental bone saw, in accordance with at least one embodiment;

FIG. 16 is a schematic diagram of a medical bone saw, in accordance with at least one embodiment;

FIG. 17 is a schematic diagram of a bone drill, in accordance with at least one embodiment;

FIG. 18 is a schematic diagram of two surgical handpieces, in accordance with at least one embodiment;

FIG. 19 is a schematic diagram of a high-torque medical tool, in accordance with at least one embodiment;

FIG. 20 is a schematic diagram of a morcellator system, in accordance with at least one embodiment;

FIG. 21 is a schematic diagram of a craniotome handpiece, in accordance with at least one embodiment; and

FIG. 22 is a schematic diagram of a medical shaver, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

Embodiments of the inventions disclosed herein are described in the context of ultrasonic dental scalers because they have particular utilities in this context. However, the inventions disclosed herein can be used in other contexts as well, such as other types of dental tools, surgical tools, and other medical devices.

In the following detailed description, terms of orientation such as “upper,” “lower,” “longitudinal,” “horizontal,” “vertical,” “lateral,” “distal”, “proximal”, “midpoint,” and “end” are used herein to simplify the description in the context of the illustrated embodiments. Because other orientations are possible, however, the present inventions should not be limited to the illustrated orientations. Those skilled in the art will appreciate that other orientations of various components described herein are possible.

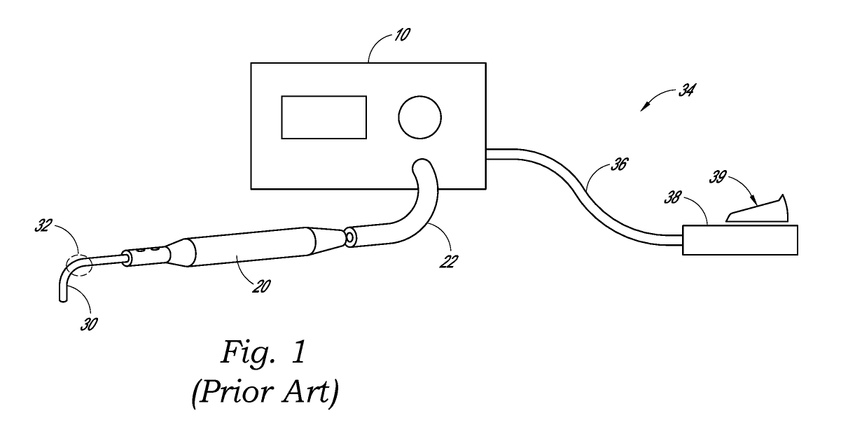

FIG. 1 schematically illustrates a prior art ultrasonic scaler. It consists of a main unit 10 which can be considered as a sonic or ultrasonic driver, a handheld piece 20 and the ultrasonic tip assembly 30. Some prior art ultrasonic tip assemblies have a hollow canal to guide the water to the opening in the concave region 32 of the insert so that the water can clean the examined region. The hollow canal may or may not reach the tip of the assembly 30, therefore water may not be able to reach the tip. Such traditional assemblies 30 are not able to deliver the laser light to a tooth region.

Such prior art ultrasonic scaler systems can include a foot pedal actuator assembly 34 including a control line 36 and a foot pedal 38. In this type of configuration, the foot pedal actuator assembly 34 includes a switch (not shown) in the foot pedal assembly 38. The switch is actuatable by a moveable pedal member 39 which is pivotably mounted relative to a base of the foot pedal assembly 38. The control line 36 can include one or more electrical wires configured to cooperate with the switch within the foot pedal assembly 38 and for providing an on/off signal and/or functionality for the main unit 10. As such, the main unit 10 is configured to turn or turn off a sonic or ultrasonic signal delivered to the handheld piece 20. In some systems, ultrasonic vibrations are conducted through an air passage to the tip assembly 30. In piezoelectric systems, electrical signals are delivered to a piezo electric transducer in the handheld piece 20. Thus, during use, a user can hold the handheld piece 20 placing the ultrasonic tip assembly 30 into proximity and/or contact with a patient's anatomy and use the foot pedal control assembly 34 for turning on or turning off the delivery of ultrasonic signal to the assembly 30.

The handheld piece 20 is connected to the main unit 10 with a connector hose 22. The connector hose 22 can include an ultrasonic delivery channel (delivering ultrasonic vibrations conducted by air or in the form of electrical signals to a piezoelectric transducer), and optionally a water delivery channel. The foot pedal assembly 34 can be used to control the actuation of the ultrasonic signal to the tip assembly 30 and/or water delivery to the tip assembly 30.

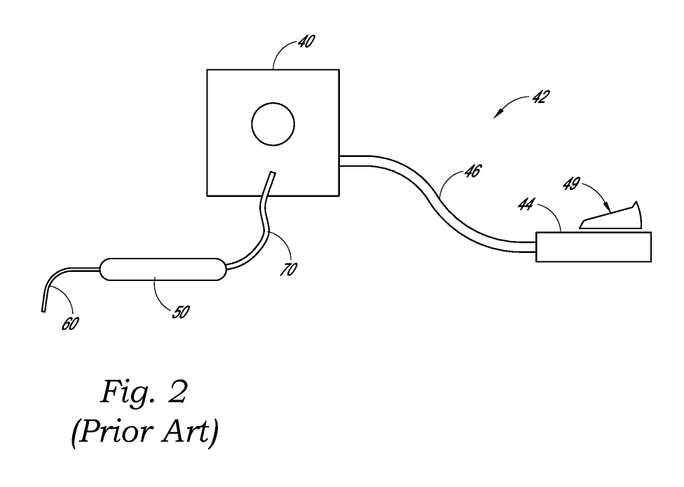

FIG. 2 is a schematic illustration of a prior art laser curettage device. The laser curettage device of FIG. 2 includes a laser driver unit 40, an optical fiber unit 70, handheld piece 50, and a fiber probe 60. Such a curettage device can be foot-pedal controlled. For example, the laser curettage prior art system can include a foot pedal assembly 42 including a foot pedal unit 44 connected to the driver 40 with a foot pedal control line 46. The foot pedal unit 44 can include a user actuatable foot pedal member 49. As such, the foot pedal control unit 42 can be used to trigger the driver 40 to turn on or turn off light energy, such as laser light energy, delivered to the fiber 70 and ultimately to the fiber 60.

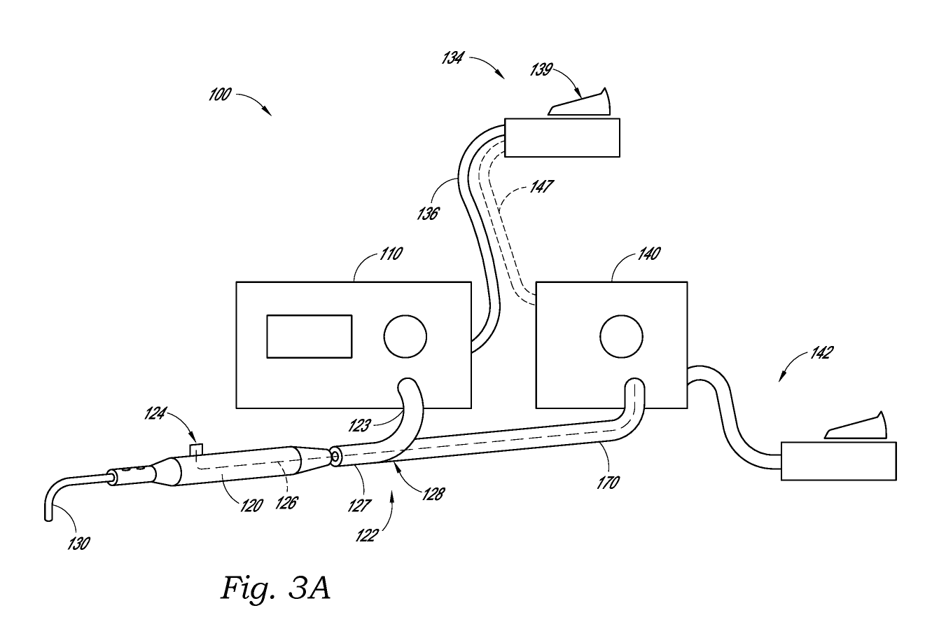

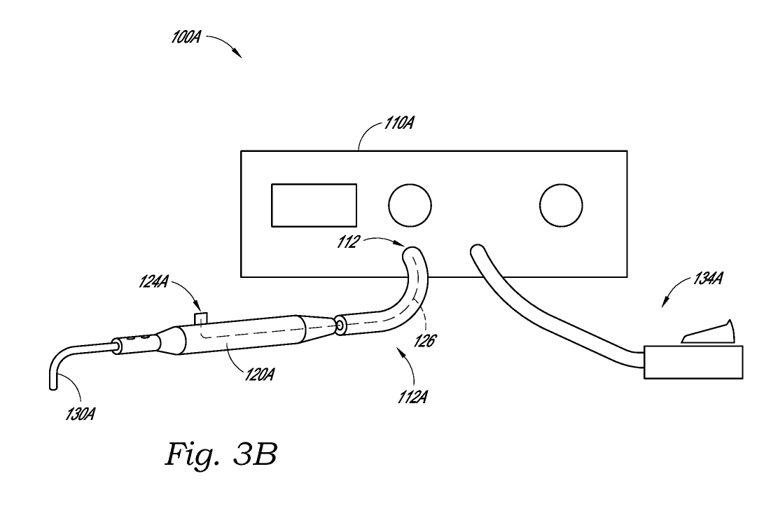

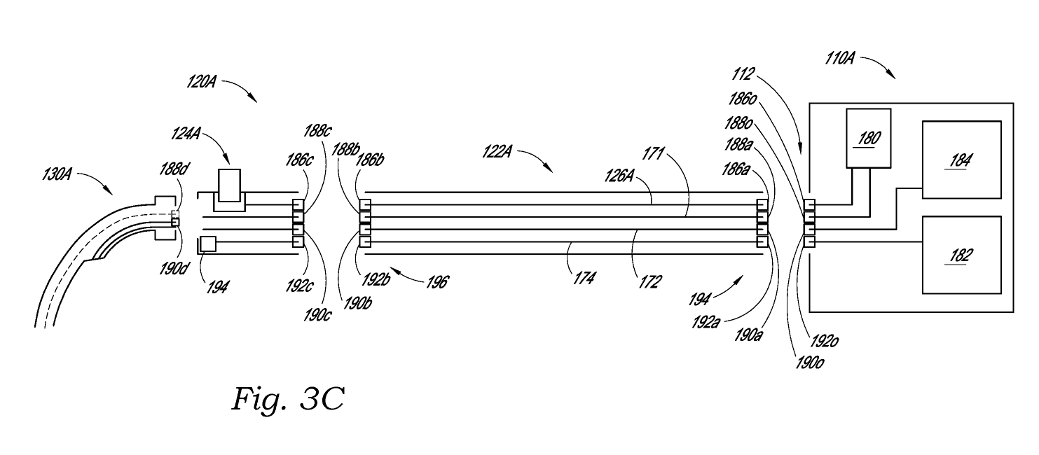

FIG. 3 illustrates an embodiment of a dental scaler with light therapy system 100 in accordance with an embodiment. Some components of the system 100 are described with the same reference numerals used for identifying portions of the systems shown in FIGS. 1 and 2 because they can have similar construction, except as described below.

As shown in FIG. 3A, the system 100 includes a driver unit 110, a light therapy driver unit 140, a connector assembly 122, a handheld piece 120, and a scaler tip assembly 130.

The driver unit 110 can be constructed in accordance with the driver unit 10 of FIG. 1, and can include a foot pedal control assembly 134. Similarly, the light therapy driver unit 140 can be in the form of the light therapy unit 40 of FIG. 3, and can include a foot pedal control assembly 142.

Optionally, the foot pedal control assembly 134 can be connected to both the driver unit 110 with a control line 136 as well as an optional light therapy control line 147. In some embodiments, the foot pedal control assembly 134 can include a single pedal 139 operably connected via the control lines 136 and 147 to the driver units 110 and 140, respectively, for turning on the sonic signal from the driver unit 110 and the light from the light therapy driver unit 140 through a single operation. In at least one embodiment, the foot pedal control assembly 134 is capable of turning on the driver unit 110 and the light therapy driver unit 140 independently (such that one or both of the driver unit 110 and the light therapy driver unit 140 may be selectively powered on and off) or simultaneously. This functionality may be incorporated in any of the other embodiments/configurations described herein.

Optionally, the handheld piece 120 can include an input device 124 accessible on an outer surface of the handheld piece 120. For example, the input device 124 can be in the form of a user actuatable button, or any other type of input device. The input device 124 can be connected to the light therapy driver unit 140 with a control line 126 extending along and/or through the handheld piece 120 and the connector line 122, into the light therapy device 140, for performing essentially the same function as the foot pedal assembly 142. In at least one further embodiment, each of the driver unit 110 and the light therapy driver unit 140 may be selectively powered on and off using any other mechanism (i.e., hardware and/or software) now known or later developed. This functionality may be incorporated in any of the other embodiments/configurations described herein.

In some configurations, the connector line 122 can be bifurcated, including a common end 127 connected to an input end of the handheld piece 120, and a bifurcated portion 128 at which location the connector line 122 is split into a sonic driver portion 123 and a light therapy connector portion 170. Other configurations can also be used.

In operation, a sonic signal from the sonic driver unit 110 can be delivered to the handheld piece 120, and then to the ultrasonic scaler tip assembly 130. Light, such as laser light, from the light therapy driver unit 140 can also be delivered to the handheld piece 120 through the connector portion 170, which can contain an optic fiber or channel or other form of light guide.

As such, ultrasonic signal and light therapy features are integrated and can be simultaneously (or alternatingly) delivered to the handheld piece 120 via the ultrasonic tip assembly 130. Thus, the system 120 can reduce potentially pathogenic microorganisms or other infective matter in the air and other targeted entities, providing a safer working environment. Furthermore, by delivering the light therapy features through the tip assembly 130 in such embodiments, the light therapy features are not susceptible to any physical blockage—i.e., the light therapy features are capable of reaching anywhere that the tip assembly 130 is able to reach, without being blocked by a tooth, tissue, device tip or other dental tools, for example. Additionally, with both ultrasonic signal and light therapy features being delivered through the tip assembly 130 in such embodiments, the relative bulk of the handheld piece 120 is minimized, thereby enabling the tip assembly 130 to reach certain target areas that would otherwise be too cumbersome (or even impossible) for relatively bulkier tools to reach—such as all locations within periodontal pockets. This arrangement and functionality may be incorporated in any of the other embodiments/configurations described herein.

In at least one embodiment, where the light therapy features comprise laser light, the laser light has a wavelength that ranges from 780 nanometers to 980 nanometers; a pulse rate that ranges from 100 microseconds to 100 milliseconds; an energy rate that ranges from 1 millijoule per pulse to 10 millijoules per pulse; a frequency that ranges from 100 hertz to 1,000 hertz; and a total power that ranges from 1 watt to 6 watts. In at least one such embodiment, the laser light preferably has a wavelength of 810 nanometers, a pulse rate of 1 millisecond, an energy rate of 6 millijoules per pulse, a frequency of 500 hertz, and a total power of 3 watts. In at least one embodiment, such preferred parameters enable the laser light to destroy bacteria to depths of 2-3 millimeters into the periodontal tissues of the patient. Additionally, such parameters avoid the need for photosensitizers in the patient's mouth (or elsewhere in the body), which eliminates the associated risks of an adverse reaction to the photosensitizer and also avoids the need for post-rinse or bleaching solutions to remove the discoloring/staining effects of intra-oral photosensitizers. Avoiding the need for photosensitizers also greatly reduces the typical treatment time. For example, in a study that was conducted by Applicants in connection with treating periodontal pockets for dental prophylaxis and aerosol bacterial reduction (which can occur simultaneously when using an exemplary embodiment of the present invention), typical treatment time per pocket using an exemplary embodiment of the present invention was less than ten seconds per pocket. By comparison, performing the same type of treatment using a process that requires photosensitizers—such as anti-microbial photo dynamic therapy (“aPDT”)—can require up to one minute per pocket. This is because such prior art photosensitizer processes require the teeth to be cleaned and the pocket debrided prior to the application of the photosensitizer, which can be laborious and involves infusing each pocket with the dye separately; then the laser is used for photodisinfection, followed by dye/photosensitizer removal by rinsing and residual stain removal through bleaching or similar. Additionally, in at least one embodiment, such parameters enable the laser light to operate in many areas of the body where aerosol is generated by medical procedures—where it would be impossible, impractical or unsafe to place a photosensitizer for such procedures. The laser light is also able to be used in all sites throughout the mouth, where there are both healthy and diseased tissue—whereas devices that utilize photosensitizers are only capable of being used on diseased/inflamed sites—and is able to prevent cross-contamination or transfer of periodontal pathogens and/or infectious aerosols from a site of disease to a healthy one during routine oral prophylaxis.

In at least one embodiment, the ultrasonic tip assembly 130 is configured to deliver both light and water to a desired target area, as well as ultrasonic energy. For example, the connector 122 can be configured to deliver water from the sonic driver unit 110, to the handpiece 120, and to the ultrasonic tip assembly 130. In at least one embodiment, the ultrasonic tip assembly 130 can receive light energy from the light therapy driver unit 140 and water from the sonic driver 110 in a pulsed, alternating fashion, so as to avoid inactivating the photobactericidal effect by light absorption in the water spray. This functionality may be incorporated in any of the other embodiments/configurations described herein. The system 100 can also reduce the possible transfer of periodontal pathogenic bacteria and other infective matter from a diseased pocket to healthy sulcus.

In some embodiments, the ultrasonic tip assembly 130 can be color coded or graduated, providing an indicia indicating a size of a referenced dimension of the tip assembly 130. Such a color coding technique can allow a clinician or practitioner to have a convenient means for measuring or estimating a measurement of an area, such as an anatomical structure or defect of a patient. For example, if an anatomical structure such as a pocket, is smaller than an ultrasonic tip assembly 130 then being used by the clinician, or depending on the size and tenacity of the calcified deposits, the clinician can find a smaller size tip, indicated by color coding of the tip, switch to a smaller size tip by installing onto the handheld piece 120, and continue the procedure. In at least one such embodiment, the color coding is positioned on the tip assembly 130 and configured for identifying the depth of the tip assembly 130 as it is inserted into a sulcus/pocket, which assists the clinician in identifying their working depth within such a sulcus/pocket or other structure and whether or not they are accessing the desired location such as the base thereof.

Additionally, the system 100 can provide a further advantage in that a dental professional can use a single device to perform both scaling and root planning, remove any remaining soft tissue tags, reduce bacteria levels, and promote wound healing.

FIG. 3B illustrates a modification of the embodiment of FIG. 3A. In the embodiment of FIG. 3B, the modified embodiment is identified generally by the reference numeral 100A. Components, parts, and features, and functionality of the system 100A can be similar or the same as those of the system 100 described above and thus corresponding components have been identified with the same reference numeral, except that a letter “A” has been added thereto.

With reference to FIG. 3B, the system 100A can include an integrated sonic and light driver device 110A which includes the components and functionalities of both the driver unit 110 and the light therapy driver unit 140 of the system 100 described above.

Optionally, the integrated driver unit 110A can include a single output port 112 including outputs for both ultrasonic signal and light for delivery to the handheld device 120A. Additionally, the control line 126A can extend from the input device 124A to the integrated driver 110A. As such, the integrated driver unit 110A can be configured to deliver any one or any combination of ultrasonic signal, water, and light for delivery to the tip assembly 130A.

The integrated driver unit 110A can receive a control signal from the input 124A through the control line 126A. The driver unit 110A can be configured to use the signal from the control line 126A to control any one or any combination of delivery of sonic energy and/or light. Similarly, the control assembly 134A can be connected to the integrated driver unit 110A and can be used to control any one of or any combination of sonic energy and light delivered to the ultrasonic tip assembly 130A.

FIG. 3C is a schematic diagram illustrating the connections between the integrated driver unit 100A and the ultrasonic tip assembly 130A. As shown in FIG. 3C, the integrated driver unit 110A can include a light source 180, sonic energy source 182, and a water source 184 (including, in at least one embodiment, an attachment hose to utilize a stationary water source such as that within a dental operatory). Additionally, the integrated driver unit 110A can include a light control connector 186o, a light energy connector 188o, a water supply connector 190o, and a sonic energy connector 192o.

The connector assembly 122A can include a control line 126A, a light optical fiber 170, a water channel 172, and a sonic energy conduit 174. Additionally, the connector assembly 122A can include an input end 194 and an output end 196. The input end 194 can be configured to connect to the output port 112 of the integrated driver unit 110A. For example, the input end 194 can include corresponding connectors 186a, 188a, 192a, 190a. As such, the input end 194 of the connector assembly 122A can connect to the connector 112 with the connectors 186a, 188a, 192a, 190a, connecting with the connectors 186o, 188o, 190o, 192o, respectively.

Similarly, the output end 196 of the connector assembly 122A can include connectors 186b, 188b, 190b, and 192b. Additionally, the handheld piece 120A can include corresponding connectors 186c, 188c, 190c, and 192c. As such, the input end of the handheld piece 120A can connect to the output end 196 of the connector assembly 122A, with the connectors 186c, 188c, 190c, 192c connecting with the connectors 186b, 188b, 190b, 192b, respectively.

The connector 186c can provide electrical connection to the input device 124A for providing the signal to the light energy source 180.

The connector 186c can provide an optical connection to the ultrasonic scale or tip assembly 130A, described in more detail below.

The connector 190c can provide a connection for water from the water source 184 to the ultrasonic tip assembly 130A. Finally, the connector 192c can provide a fork connection and transfer of sonic energy from the sonic energy source 182 to a sonic actuator 194 within the handheld piece 120A.

The ultrasonic tip assembly 130A can include an optical connector 188d and a water connector 190d. As such, the ultrasonic tip assembly 130A can receive light energy from the light source 180 of the connector, 188d and water from the water source 184 through the water connector 190b. The various connecters described above can be in the form of any known connector, including butt connectors, male-female connectors, or other types of connectors well known in the art for various types of connecting functionalities.

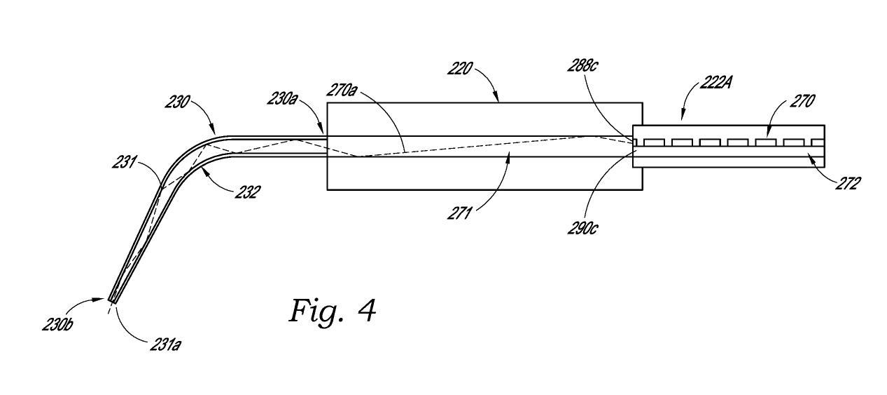

FIG. 4 illustrates a modification of the handheld piece 120, identified generally by the reference number 220. Parts, components, features, and functionality of the handheld piece assembly 220 are similar or the same as the handheld pieces 120, 120A described above, are identified generally with the same reference numerals except that 100 has been added thereto.

With reference to FIG. 4, handpiece 220 can be connectable to connector assembly 222A with connectors 288C for receiving light from the fiber 270 and the connector 290C for receiving water through a water channel 272 within the connector assembly 222A. The handheld piece 220 can include a central passage 271 configured to guide both light and water to the ultrasonic tip assembly 230. With such an embodiment, both light, such as laser light from the fiber 270 and water from the channel 272 are fed to the handheld piece 220 and then to the ultrasonic tip assembly 230. The inner surface of the passage 270 can be smooth with a high reflectivity sufficient to guide light, such as laser light, to the ultrasonic tip assembly 230 with sufficient efficiency for bacterial load reduction results.

In some embodiments, the proximal end 230a of the ultrasonic tip assembly 230 can be engaged to the distal end of the handheld piece 220 with any type of engagement configuration, such as a threaded engagement, butt connector, male-female connector, or any type of connector known in the art. Some prior art devices use threaded connections, and such a connection can be used in the embodiment of FIG. 4.

In some embodiments, the ultrasonic tip assembly 230 includes an internal passage 231 that is configured to guide both light and water to a distal end 230b of the ultrasonic tip assembly 230. The distal end 230b of the ultrasonic tip assembly 230 is configured to be pressed against patient anatomy, such as teeth and/or gums, for scaling in the manner well-known in the art. The distal end 230b, however, can include an aperture 231A configured to allow light and/or water to be discharged from the distal end 230B. Thus, as illustrated in FIG. 4, the light represented by the dashed line 270A is guided through the passage 271, and through the passage 231A, to the aperture 231A. Thus, during operation, both light and water can be discharged from the distal end 230b of the ultrasonic tip assembly 230. In at least one embodiment, the ultrasonic tip assembly 130A can receive light energy from the light source 180 and water from the water source 184 in a pulsed, alternating fashion, so as to avoid inactivating the photobactericidal effect by light absorption in the water spray.

Similarly to the passage 271, the passage 231 can include sufficient smoothness and reflectivity to guide light, such as laser light, to the aperture 231A with sufficient efficiency that the light discharged from the aperture 231A has sufficient intensity so as to provide desired bacterial reduction. For example, using a typical power output setting of a known laser curettage device, the passage 231 can have a 50% reflectivity or more and sufficiently guide laser light out of the tip assembly 230 for bacterial load reduction.

In some embodiments, as illustrated in FIG. 4, an internal dimension, such as a diameter, of the passage 231 can reduce gradually from the proximal end 230A to the distal end 230B. Such a gradually reducing inner dimension of the passage 231 can provide for more light to be delivered to the distal end 230B and discharged through the aperture 231A.

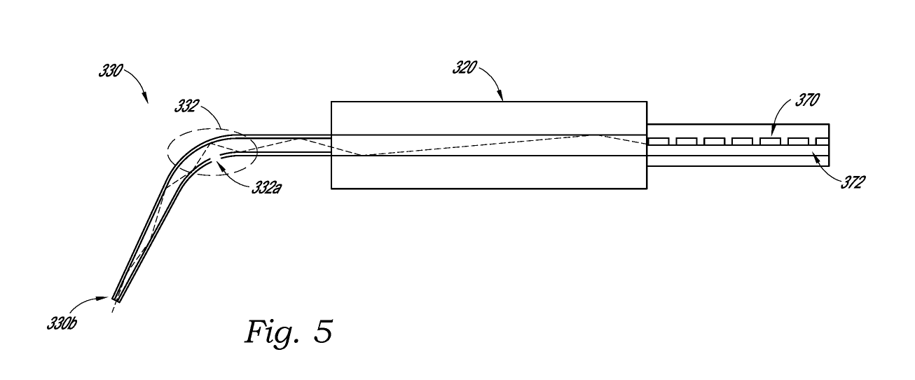

FIG. 5 illustrates yet another modification of the handheld piece 120, identified generally by the reference numeral 320. Parts, components, features and functionality of the handheld piece 320 that are similar or the same as the previously described embodiments of the handheld piece described above are identified with the same reference numeral, except that 200 has been added thereto.

As shown in FIG. 5, the ultrasonic scaler tip assembly 330 includes an additional aperture 332a positioned approximately in the concave portion 332. The aperture 332 is defined in the passage 331 and is configured to allow the discharge of water from the internal passage 331 at a position spaced away from the distal portion 330b.

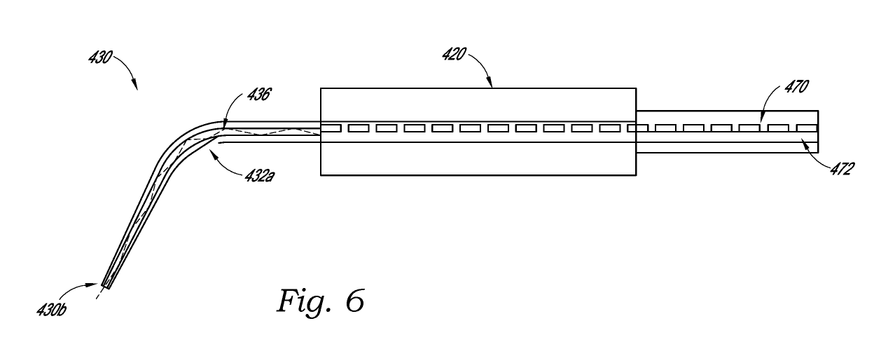

FIG. 6 illustrates yet another modification of the handheld piece 120, identified generally by the reference numeral 420. Parts, components, features, and functionality of the handheld piece 420 are identified with the same reference numerals used above with regard to the system 100, except that 300 has been added thereto.

As shown in FIG. 6, an optical fiber 436 is disposed in the tip assembly 430 to deliver laser light to the distal end 430b of the tip assembly 430. Using such an additional portion of optical fiber 436 in the tip assembly 430 can provide an optional additional advantage of improving light transmission efficiency as compared to using a hollow passage, such as in the embodiments of FIGS. 4 and 5. Optionally, an opening 432a in the concave region of the insert can be configured to deliver water for cleaning purposes during dental procedures such as scaling. In some embodiments, the laser light from the laser fiber 470 is directly coupled into the optical fiber 436 in the insert 430.

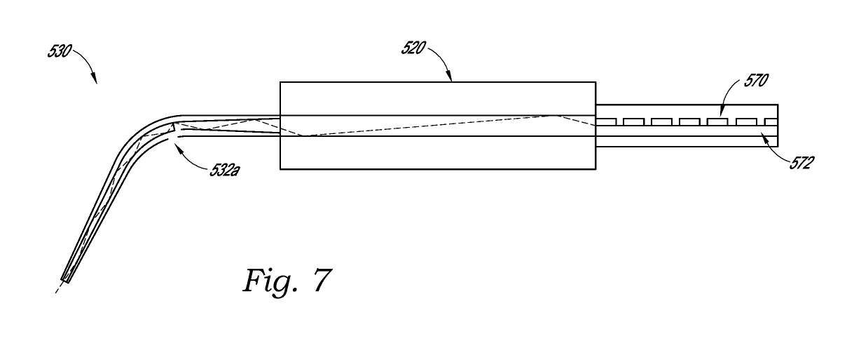

FIG. 7 illustrates yet another modification of the handheld piece 120, identified generally by the reference numeral 520. Parts, components, features, and functionality of the handheld piece 520 are identified with the same reference numerals used above with regard to the system 100, except that 400 has been added thereto. In this embodiment, the optical fiber 536 only extends from the tip to the opening 532a of the tip assembly 530.

The embodiments of FIGS. 4-7 have internal components to couple the laser light from the fiber 270, 370, 470, 570 through the corresponding handheld piece to the optical fiber 436, 536 or a hollow canal within the tip assembly. Any known art light coupling hardware or methods can be used inside the various embodiments of the handheld pieces to couple the laser light to the hollow canal or optical fiber in the tip assembly—for example, direct fiber contact in FIG. 6, hollow light pipe in FIGS. 4, 5 and 7. Other methods, such as the coupling lens can be used as well. All those coupling methods can be optimized using well known techniques to maximize the coupling efficiency for the current invention. The fibers for the lasers in embodiments in FIGS. 4-7 can be connected to different laser sources.

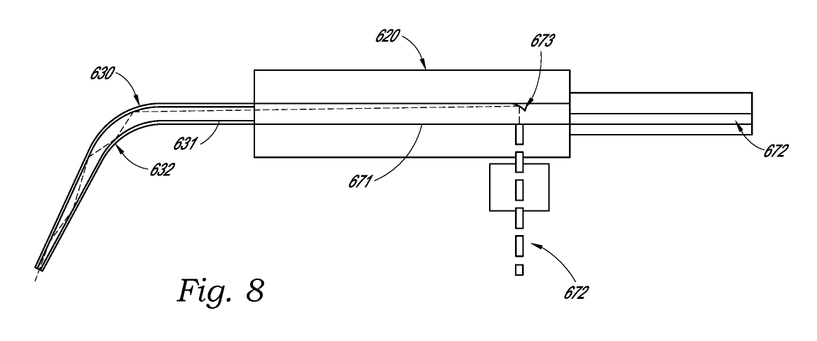

FIG. 8 illustrates yet another modification of the handheld piece 120, identified generally by the reference numeral 620. Parts, components, features, and functionality of the handheld piece 520 are identified with the same reference numerals used above with regard to the system 100, except that 600 has been added thereto.

As shown in FIG. 8, in some embodiments, light coupling can be accomplished by a reflector 673 disposed in the passage 671 within the handheld piece 620. The light from the laser fiber 672 can be coupled into the hollow canal 631 of the tip assembly 630 directly by the concave reflector 673. In some embodiments, the light can be introduced into the passage 671 by a fiber 672 connected to the passage 671 at an oblique or perpendicular angle. By using the different fiber connecting to different laser sources, dental professionals can easily perform different procedures with different laser wavelengths.

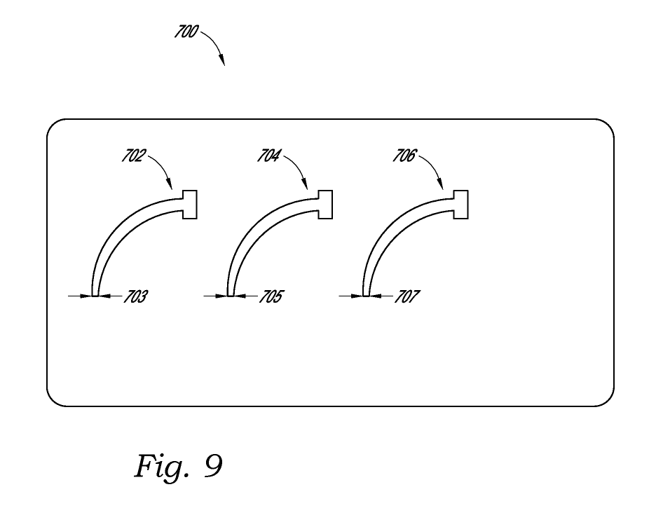

FIG. 9 is a schematic illustration of a kit 700 including a plurality of dental scaler tip members 702, 704, 706 having different widths 703, 705, 707 at their respective distal ends. Each of the dental scaler tip members 702, 704, 706 can have different colors, and as such can be considered as being color-coded according to the widths 703, 705, 707. In some embodiments, the different widths can be offset from each other by predetermined amounts such as 0.5 mm, 1 mm, 2 mm, or other magnitudes. Additionally, as mentioned above, tips may also be coded according to the amount and tenacity of the calcified deposits present on the teeth. The kit 700 can include any type of sterilizable container for containing a plurality of dental scaler tip members.

The following further non-limiting examples (in addition to the above-discussed non-limiting examples) are provided for illustrative purposes only in order to facilitate a more complete understanding of representative embodiments now contemplated. These examples are intended to be a mere subset of all possible medical devices which may utilize the laser therapy capabilities disclosed herein. Thus, these examples should not be construed to limit any of the embodiments described in the present specification.

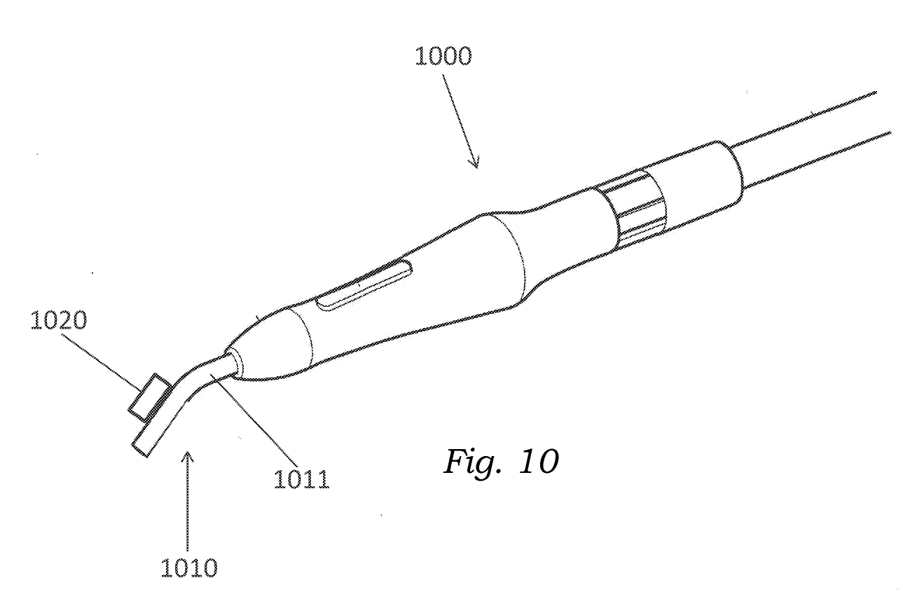

FIG. 10 illustrates an embodiment of a laser-enabled instrument in the form of an air polishing unit 1000 that uses a combination of air and water to deliver a stream of fine abrasive powder or liquid onto the tooth surface. Abrasives have the effect of removing stains and plaque from the outer surface of the tooth. A handle portion of the air polishing unit 1000 can be configured to be graspable by a user's hand and manipulable by an operator. The handle can be configured to be coupled with a connection hose or a wire for delivering power, air, liquid and/or other materials to the air polishing unit 1000. A distal end of the air polishing unit 1000 can include a blower 1010.

The blower 1010 can be configured to have an internal conduit 1011 through which the air and/or liquid can be sprayed from the air polishing unit 1000. In addition, in some embodiments the air polisher unit 1000 can include a light guide or optical cable/fiber that extends from a proximal end of the blower 1010 towards the distal end. The light guide can extend through the internal conduit or another hollow space disposed within the blower 1010 to the distal end of the blower 1010. Thereby the light guide can be configured to output a laser light such that it is pointed or directed in the same direction as the liquid and/or air that is output from the distal end of the blower 1010 of the air polishing unit 1000. For example, in some embodiments the laser output can be disposed within five millimeters of the distal end and/or be configured with a lens having a focal length of five millimeters or less. Thus the laser light can shine against a working surface or tissue of a patient (e.g., a tooth) to treat that surface using the laser light, as described above.

In some embodiments, at least a portion of the light guide can extend from the housing and along a portion of an outer surface of the blower 1010 to a laser output 1020. The laser output 1020 can be configured to output the laser light in the direction of the distal end of the blower 1010. For example, in some embodiments the laser output can be disposed within five millimeters of the distal end and/or be configured with a lens having a focal length of five millimeters or less. The laser output 1020 can be oriented such that it is pointed or directs the laser light in the same direction as the liquid and/or air is output from the distal end of the instrument air polishing unit 1000. Thus the laser light can shine against a working surface or tissue of a patient (e.g., a tooth) to treat that surface using the laser light from a laser light generator, as described above. In some embodiments, the laser output 1020 can be removably or permanently coupled or otherwise attached onto the outside surface of the distal end of the blower 1010. In at least one embodiment, the laser output 1020 is positioned and configured in either a side-firing, forward-firing, or a combined side- and forward-firing light delivery arrangement in order to achieve light delivery to desired targets positioned in front of the distal end of the instrument and/or lateral to the distal end of the instrument, for achieving full and rapid coverage of the side and floor structures of the working surface or tissue of a patient, such as a periodontal pocket. In such embodiments, the location of the laser output 1020 is preferably positioned to maximize aerosol impact while avoiding heating the working surface or tissue of the patient. This functionality may be incorporated in any of the other embodiments/configurations described herein.

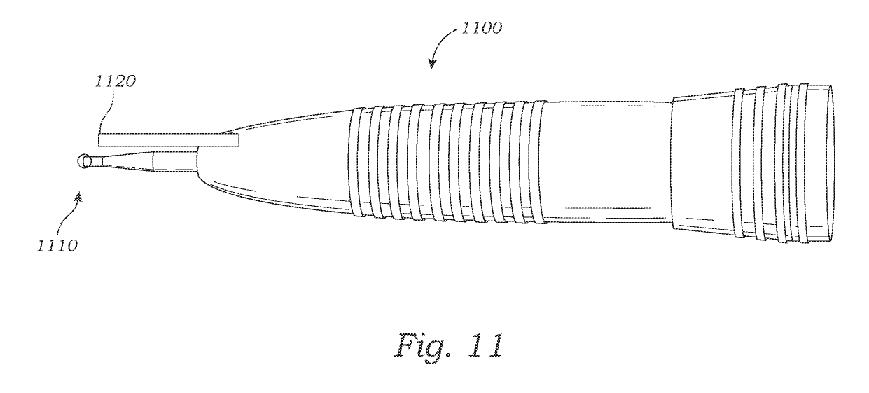

FIG. 11 illustrates an embodiment of a laser-enabled instrument in the form of an straight dental handpiece 1100 that can be used for rotating dental tools, cutting, grinding, and/or polishing. The dental handpiece 1100 can be powered using a turbine and pressurized air. A handle portion of dental handpiece 1100 can be configured to be graspable by a user's hand and manipulable by an operator. The handle can be configured to be coupled with a connection hose or a wire for delivering power, air, liquid and/or other materials to dental handpiece 1100. A distal end of the dental handpiece 1100 can include a tool 1110.

In some embodiments, at least a portion of a light guide can extend from the housing and along a portion of an outer surface of the tool 1110 to a laser output 1120. The laser output 1120 can be configured to output the laser light in the direction of the distal end of the tool 1110. For example, in some embodiments the laser output can be disposed within five millimeters of the distal end and/or be configured with a lens having a focal length of five millimeters or less. The laser output 1120 can be oriented such that it is pointed or directs the laser light in the same direction as the liquid and/or air is output from the distal end of the instrument dental handpiece 1100. Thus the laser light can shine against a working surface or tissue of a patient (e.g., a tooth) to treat that surface using the laser light from a laser light generator, as described above. In some embodiments, the laser output 1120 can be removably or permanently coupled or otherwise attached onto the outside surface of the distal end of the blower 1120.

Some embodiments of the laser output 1120 (or any laser output described herein) can be coupled with a boom extending from a proximal end of the tool 1110 and/or the housing of the dental handpiece 1100. The boom can be configured to locate and/or orient the laser output 1120 to be disposed within five millimeters of the distal end and/or be configured with a lens having a focal length of five millimeters or less.

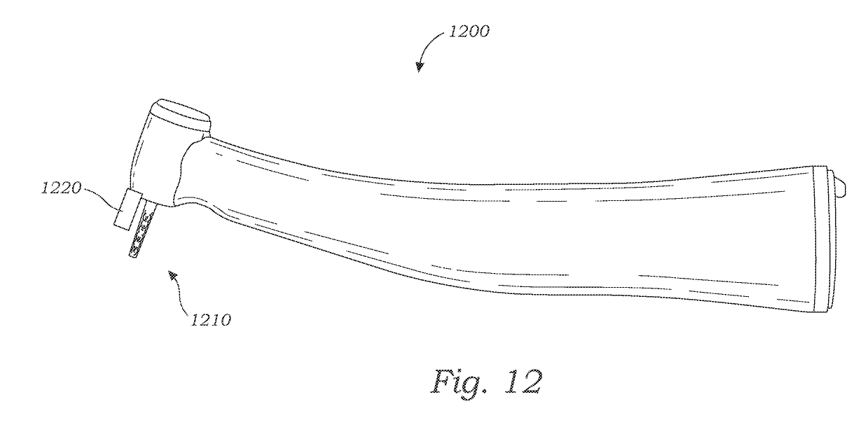

FIG. 12 illustrates an embodiment of a laser-enabled instrument in the form of an angled dental handpiece having a tool 1210 and a laser output 1220 and otherwise having a similar layout to the laser-enabled instruments described above.

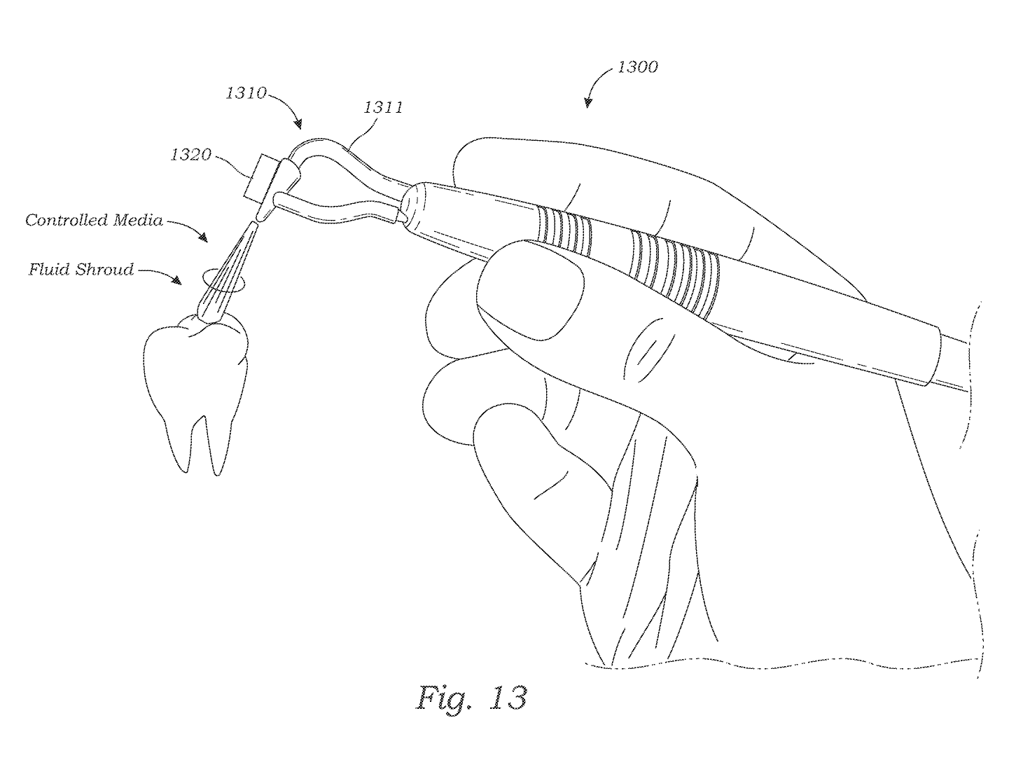

FIG. 13 illustrates an embodiment of a laser-enabled instrument in the form of an abrasion unit 1300 that uses a high-pressure stream of aluminum oxide to remove dental enamel. A handle portion of the abrasion unit 1300 can be configured to be graspable by a user's hand and manipulable by an operator. The handle can be configured to be coupled with a connection hose or a wire for delivering power, air, liquid and/or other materials to the abrasion unit 1300. A distal end of the abrasion unit 1300 can include a blower 1310.

The blower 1310 can be configured to have an internal conduit 1311 through which the air and/or liquid can be sprayed from the abrasion unit 1300. In addition, in some embodiments the abrasion unit 1300 can include a light guide or optical cable/fiber that extends from a proximal end of the blower 1310 towards the distal end. The light guide can extend through the internal conduit or another hollow space disposed within the blower 1310 to the distal end of the blower 1310. Thereby the light guide can be configured to output a laser light such that it is pointed or directed in the same direction as the liquid and/or air that is output from the distal end of the blower 1310 of the abrasion unit 1300. For example, in some embodiments the laser output can be disposed within five millimeters of the distal end and/or be configured with a lens having a focal length of five millimeters or less. Thus the laser light can shine against a working surface or tissue of a patient (e.g., a tooth) to treat that surface using the laser light, as described above.

In some embodiments, at least a portion of the light guide can extend from the housing and along a portion of an outer surface of the blower 1310 to a laser output 1320. The laser output 1320 can be configured to output the laser light in the direction of the distal end of the blower 1310. For example, in some embodiments the laser output can be disposed within five millimeters of the distal end and/or be configured with a lens having a focal length of five millimeters or less. The laser output 1320 can be oriented such that it is pointed or directs the laser light in the same direction as the liquid and/or air is output from the distal end of the instrument abrasion unit 1300. Thus the laser light can shine against a working surface or tissue of a patient (e.g., a tooth) to treat that surface using the laser light from a laser light generator, as described above. In some embodiments, the laser output 1320 can be removably or permanently coupled or otherwise attached onto the outside surface of the distal end of the blower 1310. Some embodiments of the blower 1310 can include a boom structure, as described above.

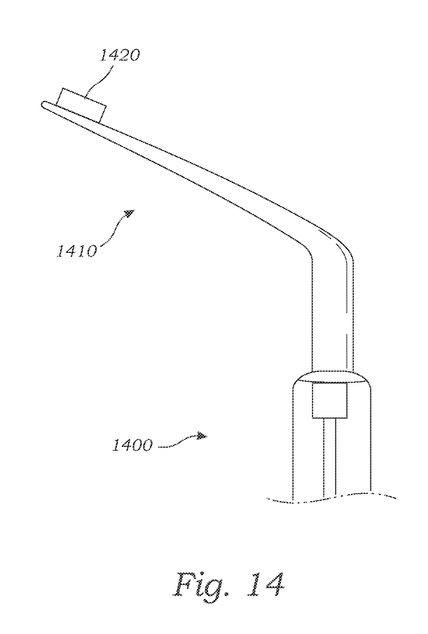

FIG. 14 illustrates an embodiment of a laser-enabled instrument in the form of an endodontic dental device 1400 having a tool 1410 and a laser output 1420 and otherwise having a similar layout to the laser-enabled instruments described above.

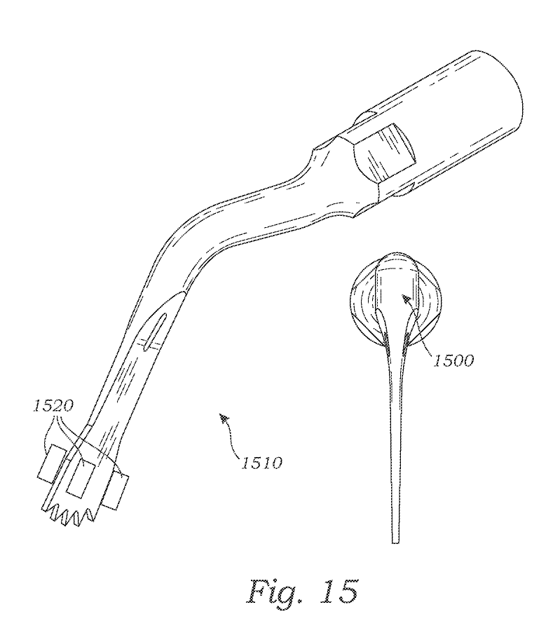

FIG. 15 illustrates an embodiment of a laser-enabled instrument in the form of an bone saw 1500 powered by a piezoelectric transducer to cut patient tissue, such as bone. A handle portion of the bone saw 1500 can be configured to be graspable by a user's hand and manipulable by an operator. The handle can be configured to be coupled with a connection hose or a wire for delivering power, air, liquid and/or other materials to the bone saw 1500. A distal end of the bone saw 1500 can include a saw 1510.

In some embodiments, at least a portion of a light guide can extend from the housing and along a portion of an outer surface of the saw 1510 to a laser output 1520. The laser output 1520 can be configured to output the laser light in the direction of the distal end of the saw 1510. For example, in some embodiments the laser output can be disposed within five millimeters of the distal end and/or be configured with a lens having a focal length of five millimeters or less. The laser output 1520 can be oriented such that it is pointed or directs the laser light in the same direction as the liquid and/or air is output from the distal end of the instrument bone saw 1500. Thus the laser light can shine against a working surface or tissue of a patient (e.g., a tooth) to treat that surface using the laser light from a laser light generator, as described above. In some embodiments, the laser output 1520 can be removably or permanently coupled or otherwise attached onto the outside surface of the distal end of the saw 1510. Some embodiments of the saw 1510 can include a boom structure, as described above.

In some embodiments, the saw 1510 can include a plurality of laser outputs 1520 to such that the laser light can be directed towards an arcuate surface of the patient tissue that contacts the saw 1510. For example, one or more laser outputs 1520 can be placed on the sides and/or faces of the saw 1510 and configured to be directed at the distal end of the saw 1510. This can more fully enable the laser treatment of all tissue that can become dislodged from the patient through the use of the bone saw 1500.

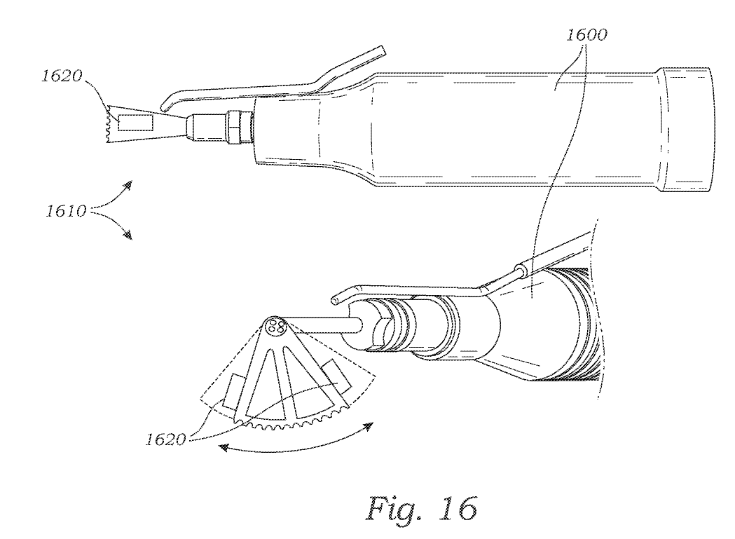

FIG. 16 illustrates an embodiment of a laser-enabled instrument in the form of a bone saw 1600 having a tool 1610 and a laser output 1620 and otherwise having a similar layout to the laser-enabled instruments described above.

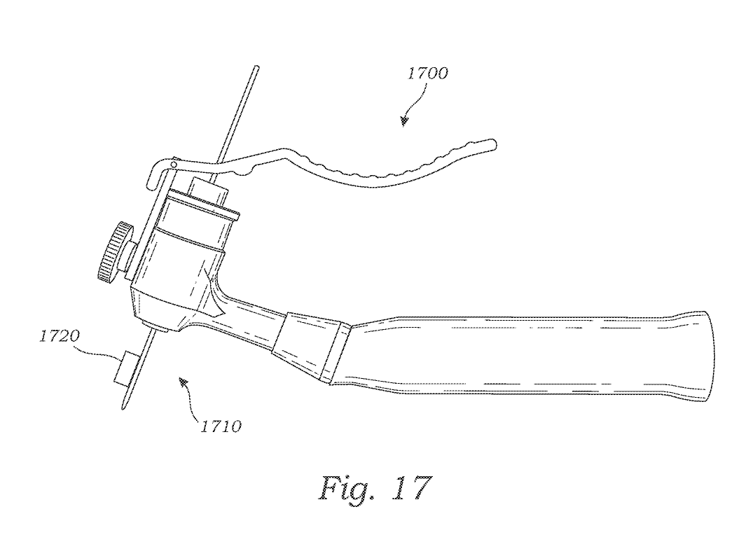

FIG. 17 illustrates an embodiment of a laser-enabled instrument in the form of an orthopedic bone drill having a tool 1710 and a laser output 1720 and otherwise having a similar layout to the laser-enabled instruments described above.

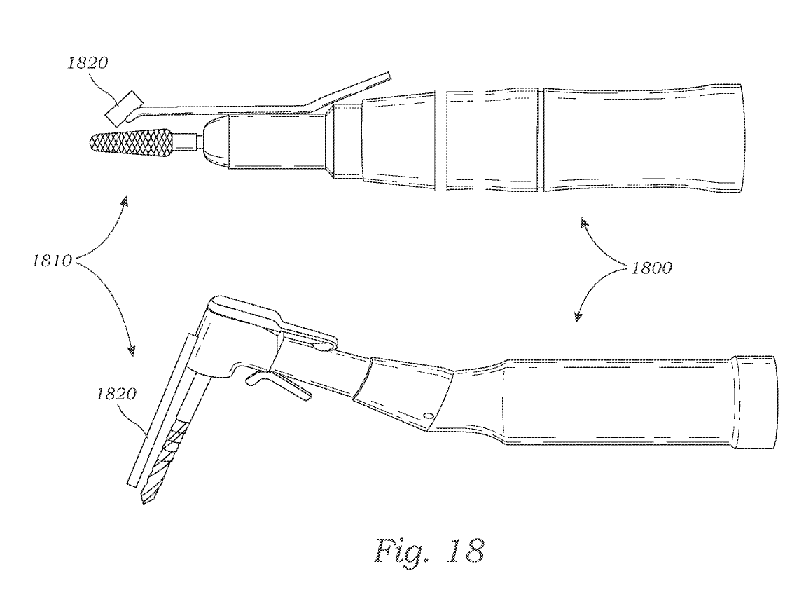

FIG. 18 illustrates an embodiment of a laser-enabled instrument in the form of a surgical handpiece having a tool 1810 and a laser output 1820 and otherwise having a similar layout to the laser-enabled instruments described above.

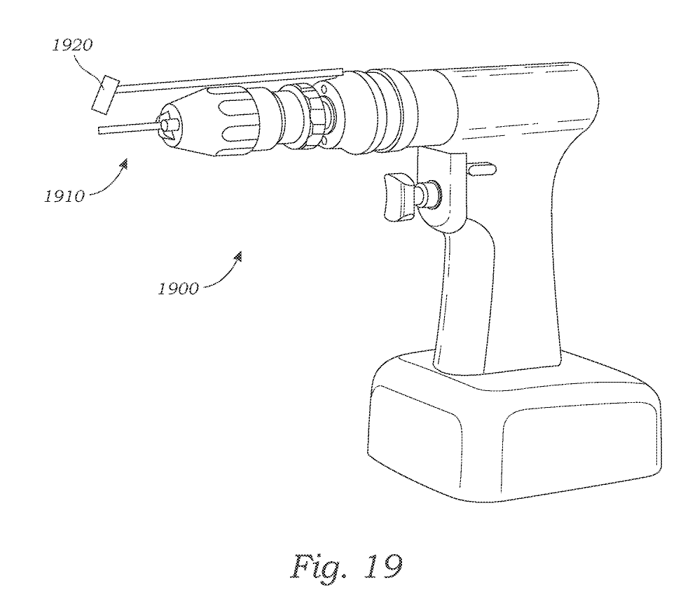

FIG. 19 illustrates an embodiment of a laser-enabled instrument in the form of a high-torque medical instrument (e.g., a bone drill) having a tool 1910 and a laser output 1920 and otherwise having a similar layout to the laser-enabled instruments described above.

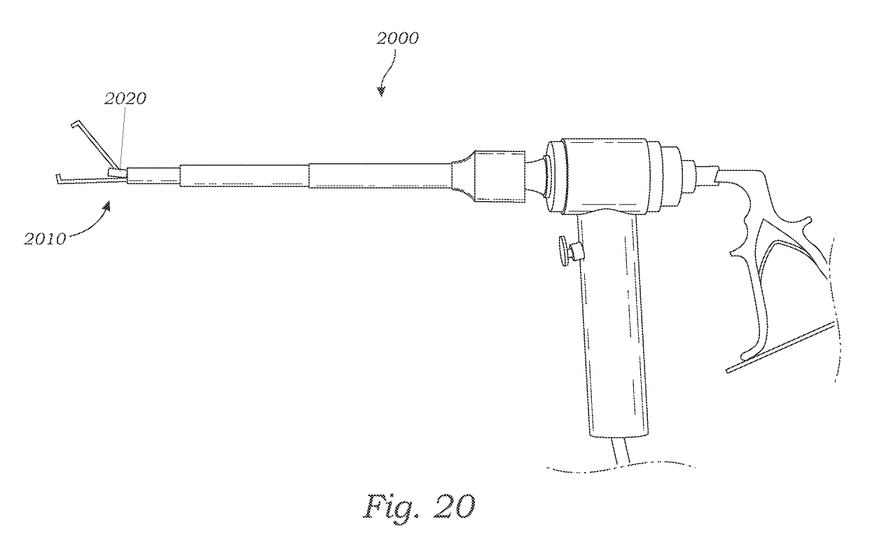

FIG. 20 illustrates an embodiment of a laser-enabled instrument in the form of a morcellator system (e.g., for laparoscopic surgery) having a tool 2010 and a laser output 2020 and otherwise having a similar layout to the laser-enabled instruments described above.

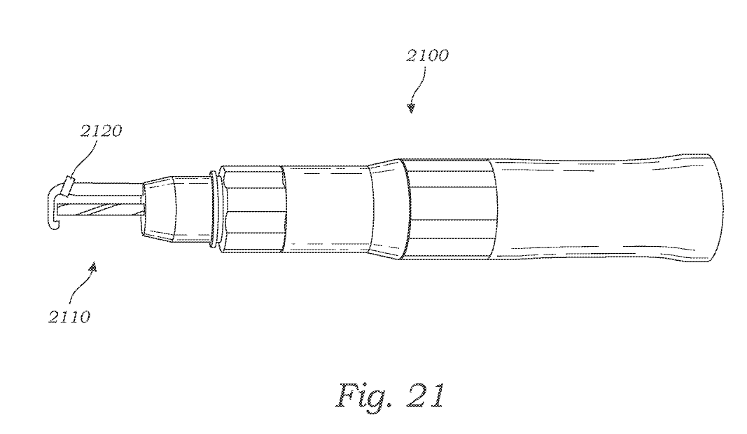

FIG. 21 illustrates an embodiment of a laser-enabled instrument in the form of a craniotome having a tool 2110 and a laser output 2120 and otherwise having a similar layout to the laser-enabled instruments described above.

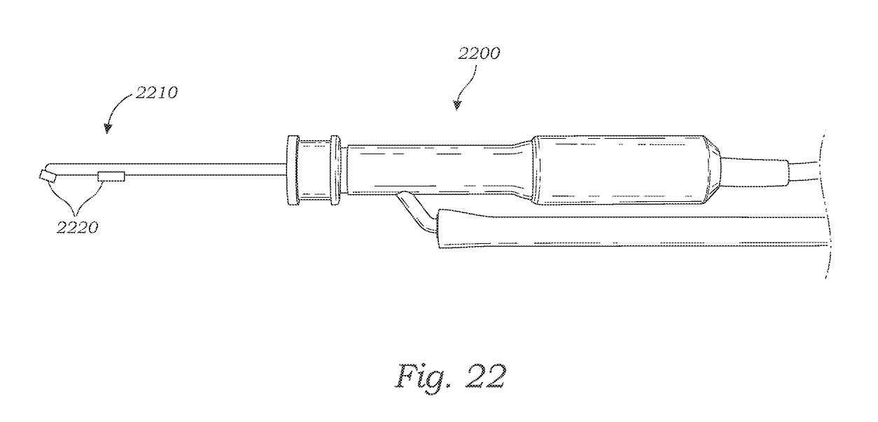

FIG. 22 illustrates an embodiment of a laser-enabled instrument in the form of a medical shaver system (e.g., for knee surgery) having a tool 2210 and a laser output 2220 and otherwise having a similar layout to the laser-enabled instruments described above.

In a pilot study that was conducted, it was found that 30-70% fewer live bacteria were released into aerosol using piezo scaling with added laser light than from using the piezo alone. This pilot study was performed in two subjects with heavy plaque and calculus presence. Using a combined piezoelectric dental scaler and laser, one upper quadrant and the opposing lower quadrant of the mouth were scaled for a period of three minutes using standard technique. During the entire process, the aerosol was collected in sterile media-containing petri dishes using standardized technique. After an interval of five minutes, the remaining quadrants were scaled using the same technique, also over three minutes, but with the piezo only. The aerosol was again collected in the same standardized fashion. The sample collected in each petri dish was stained within one hour using Live/Dead Bacterial viability stain using standard technique, and samples were imaged using a Meta System and standard fluorescence microscopy techniques. Images were evaluated using Image J, to quantify the ratio of live versus dead bacteria per mm of surface area in each sample. Samples from piezo plus laser-treated procedures averaged 30% fewer live bacteria than samples from piezo only-treated procedures.

In closing, regarding the exemplary embodiments of the present invention as shown and described herein, it will be appreciated that various types of medical devices with laser therapy capability are disclosed. Because the principles of the invention may be practiced in a number of configurations beyond those shown and described, it is to be understood that the invention is not in any way limited by the exemplary embodiments, but is generally directed to medical devices with laser therapy capability and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A medical device incorporating a light therapy system, the device comprising:
an ultrasonic driver having an ultrasonic vibration signal output port, the ultrasonic driver configured to discharge an ultrasonic frequency vibration signal from the ultrasonic vibration signal output port;
a light therapy driver having a laser light output port, the light therapy driver configured to discharge laser light from the laser light output port;
a handpiece housing having an outer surface configured to be graspable and manipulable with a user's hand;
the handpiece housing having an input assembly connected to each of the ultrasonic vibration signal output port and the laser light output port so as to receive the vibration signal from the ultrasonic driver and laser light from the light therapy driver; and
the input assembly providing an ultrasonic tip assembly configured to output each of the vibration signal and laser light, the tip assembly providing a passage in communication with and extending between the laser light output port and an operational end of the tip assembly, through which the laser light is capable of travelling and subsequently exiting the passage at the operational end of the tip assembly;
whereby, the vibration signal and laser light are capable of being simultaneously or alternatingly delivered via the tip assembly.

2. The medical device of claim 1, wherein the laser light has a wavelength that ranges from 780 nanometers to 980 nanometers.

3. The medical device of claim 2, wherein the laser light has a pulse rate that ranges from 100 microseconds to 100 milliseconds.

4. The medical device of claim 3, wherein the laser light has an energy rate that ranges from 1 millijoule per pulse to 10 millijoules per pulse.

5. The medical device of claim 4, wherein the laser light has a frequency that ranges from 100 hertz to 1,000 hertz.

6. The medical device of claim 5, wherein the laser light has a total power that ranges from 1 watt to 6 watts.

7. The medical device of claim 6, wherein the laser light has a wavelength of 810 nanometers, a pulse rate of 1 millisecond, an energy rate of 6 millijoules per pulse, a frequency of 500 hertz, and a total power of 3 watts.

8. The medical device of claim 1, wherein each of the ultrasonic driver and light therapy driver is interconnected with a foot pedal control assembly configured for selectively operating each of the ultrasonic driver and light therapy driver either independently or simultaneously.

9. The medical device of claim 1, wherein the handheld piece provides an input device accessible on an outer surface of the handheld piece, the input device configured for selectively operating each of the ultrasonic driver and light therapy driver either independently or simultaneously.

10. The medical device of claim 1, wherein the input assembly of the handpiece housing is further connected to at least one of a water source and an air source, with the tip assembly being configured to deliver both laser light and at least one of water and air via the internal passage of the tip assembly.

11. The medical device of claim 10, wherein an inner surface of the internal passage is reflective for guiding the laser light therethrough.

12. The medical device of claim 11, wherein the internal passage has an internal diameter that gradually reduces from laser light output port to the operational end of the tip assembly.

13. The medical device of claim 10, wherein the input assembly of the handpiece housing is connected to a water source, with the tip assembly being configured to deliver both laser light and water in a pulsed, alternating fashion.

14. The medical device of claim 10, wherein the air source is a blower.

15. The medical device of claim 1, wherein the tip assembly provides color coded indicia positioned and configured for identifying a working depth of the tip assembly as the tip assembly is inserted into a target area of a patient.

16. The medical device of claim 1, wherein at least a portion of the passage of the tip assembly extends a distance along an outer surface of the tip assembly and terminates at a laser output, through which the laser light exits the passage at the operational end of the tip assembly.

17. The medical device of claim 16, wherein the laser output is positioned and configured for directing the laser light in at least one of a side-firing or forward-firing direction, relative to the operational end of the tip assembly.

18. The medical device of claim 1, wherein the tip assembly is configured as at least one of a dental scaler, an air polishing unit, a dental handpiece, a dental abrasive tool, an endodontic instrument, a bone saw, a bone drill, a surgical handpiece, a high-torque medical tool, a morcellator, a craniotome, a medical shaver, and a spinal surgery angled handpiece.

19. A medical device incorporating a light therapy system, the device comprising:
an ultrasonic driver having an ultrasonic vibration signal output port, the ultrasonic driver configured to discharge an ultrasonic frequency vibration signal from the ultrasonic vibration signal output port;
a light therapy driver having a laser light output port, the light therapy driver configured to discharge laser light from the laser light output port;
the laser light having a wavelength that ranges from 780 nanometers to 980 nanometers, a pulse rate that ranges from 100 microseconds to 100 milliseconds, an energy rate that ranges from 1 millijoule per pulse to 10 millijoules per pulse, a frequency that ranges from 100 hertz to 1,000 hertz, and a total power that ranges from 1 watt to 6 watts;
a handpiece housing having an outer surface configured to be graspable and manipulable with a user's hand;
the handpiece housing having an input assembly connected to each of the ultrasonic vibration signal output port and the laser light output port so as to receive the vibration signal from the ultrasonic driver and laser light from the light therapy driver; and
the input assembly providing an ultrasonic tip assembly configured to output each of the vibration signal and laser light, the tip assembly providing a passage in communication with and extending between the laser light output port and an operational end of the tip assembly, through which the laser light is capable of travelling and subsequently exiting the passage at the operational end of the tip assembly;
whereby, the vibration signal and laser light are capable of being simultaneously or alternatingly delivered via the tip assembly.

20. A medical device incorporating a light therapy system, the device comprising:
an ultrasonic driver having an ultrasonic vibration signal output port, the ultrasonic driver configured to discharge an ultrasonic frequency vibration signal from the ultrasonic vibration signal output port;
a light therapy driver having a laser light output port, the light therapy driver configured to discharge laser light from the laser light output port;
a handpiece housing having an outer surface configured to be graspable and manipulable with a user's hand;
the handpiece housing having an input assembly connected to each of the ultrasonic vibration signal output port, the laser light output port, and at least one of a water source and an air source; and
the input assembly providing an ultrasonic tip assembly configured to output each of the vibration signal, laser light, and at least one of water and air, the tip assembly providing a passage in communication with and extending between the laser light output port and an operational end of the tip assembly, through which the laser light and at least one of water and air is capable of travelling and subsequently exiting the passage at the operational end of the tip assembly;
whereby, the vibration signal, laser light, and at least one of water and air are capable of being simultaneously or alternatingly delivered via the tip assembly.
